(19) United States
(12) Patent Application Publication
(10) Pub. No.: US 2008/0182816 A1
(43) Pub. Date: Jul. 31, 2008

(54) Methods for preparing a novel family of polysaccharride prodrugs for colonic delivery

(76) Inventors: Joemy C. Tam, Medford, NY (US); Qi Bing Mei, Xi'an (CN); Dale K.M. Choi, Medford, NY (US)

Correspondence Address: Joemy C. Tam, Bldg. 5, Suite 2, 3235 Route 112, Medford, NY, 11763, US

(21) Appl. No.: 11/657,755

(22) Filed: Sep. 17, 2007

Related U.S. Application Data
(60) Provisional application No. 60/816,169, filed on Jun. 23, 2006.

(30) Foreign Application Priority Data
Feb. 23, 2006 (CN) 200610041838.0

(51) Int. Cl. A61K 31/7072; A61K 31/715; C07H 1/00; C07H 19/04; A61P 35/00

(52) U.S. Cl. 514/50; 514/54; 536/123.1; 536/28.55

(57) ABSTRACT

This invention describes a novel family of polysaccharide prodrugs with enhanced colonic delivery advantage. The prodrugs are synthesized by chemically linking a parent compound with a specially selected polysaccharide (M.W. 105-107 Da) containing galactose residues. Its characteristics are that it is synthesized by chemically linking polysaccharides with the parent compound through different bridge links for targeted colonic delivery; that the polysaccharides contain galactose residues. Because of this, the polysaccharide component can protect the parent compound from absorption (or metabolism) in the upper gastrointestinal tract and deliver a high concentration of the bound compound to the colonic area. Upon reaching the colon, the active component of the parent drug will be released locally from the polysaccharide via enzymatic hydrolysis, allowing it to act locally for colonic disease such as inflammation or infection and/or taking advantage of the favorable microenvironment in the colon for steady and stable colonic drug absorption.

### BACKGROUND OF THE INVENTION

Oral delivery of drugs is quite common, but the physical and/or chemical conditions in the upper gastrointestinal tract may cause the drug to have poor absorption. One way to get around this is to use colonic delivery system. There are little digestive enzymes, like peptidases, in the colon which makes it a better place for drug uptake (Gibson et al., 1989 and Van den Mooter et al., 1998). There is also less intense motility and more uniform environment to allow stable and homogeneous drug absorption compared to stomach or small intestine. Also, longer retention time in the colon and its responsiveness to enhancing agents facilitate absorption of normally poorly absorbed drugs (Youan, 2004). Nevertheless, colon drug delivery is not easy to achieve. Currently, there are several ways to achieve potentially colonic delivery of drugs: pH, transit time, pressure, and bacteria in the gastrointestinal tracts.

The gastrointestinal pH values vary from the stomach to the colon (Evans et al., 1988). The value rises from about pH 1.5 in the stomach to about pH 6.0 in the proximal small intestine. Then, the value continues to augment to reach about pH 7.5 in the distal small intestine. After that, the value declines to about pH 6.4 in the cecum and gradually increases to about pH 7.0 in the distal colon (Evans et al., 1988). Eudragit®S is one of the first drugs to use gastrointestinal pH value to trigger its release (Dew et al., 1982). Using the solubility above pH 7.0 of the co-polymer of methacrylic acid and methyl methacrylate, Eudragit®S shows initially promising results in colon deliverance. However, further studies using gamma scintigraphy in healthy volunteers; the positions of disintegration of Eudragit®S are very unreliable, stretching from the proximal small intestine to the distal colon (Ashford et al., 1993). One complication is that patients with ulcerative colitis have much lower colonic pH value than their healthy counterparts, so the drug may never dissolves in the colon (Nugent et al., 2001).

The average transit time in the gastrointestinal tracts from the mouth to the rectum is about 24 hours (Wilding, 2001), but the other figures are also reported (Khosla et. at., 1989 and Tuleu et al., 2002). Depending on the drug formulation and individual fed status, the stomach has the most variable transit time ranging from a few seconds to many hours (Kaus et al., 1984; Davis et al., 1984; and Devereus et al., 1990). On the other hand, the average resident time in the small intestine is around 3 to 4 hours (Davis et al., 1986). To make full use of the gastrointestinal transit time, time-dependent drug release is manufactured to confer colonic delivery. Ishibashi's group uses scintigraphy to evaluate this time-dependent formulation and obtains highly variable results among fed and fasted healthy volunteers (Ishibashi et al., 1998). Hence, retention time is not a good way to target colon specificity.

The gastrointestinal pressure is produced through muscle contraction along the tract. The pressure is lower in the stomach and the small intestine because of the presence of more fluids. Absorption of fluids appears in the distal gut where higher pressure is generated to rupture the drug. This pressure-controlled colon delivery system is tested in human (Hu et al., 2000). Using gamma scintigraphy, the same group further characterizes the potential of this colonic delivery system (Hu et al., 2000). More testing is needed to validate the merit of this system.

The colon houses more than 400 various bacterial species and has 10⁷ times more bacteria compared to the small intestine (Finegold et al., 1983 and Abu Shamat, 1993). These bacteria secrete enzymes to metabolize non-digest carbohydrates and proteins from the upper gastrointestinal tract (Cummings et al., 1989). These enzymes also help digest natural polysaccharides that can be used as a drug carrier. Many examples are shown to use this polysaccharide-bacterial enzyme system that holds a great promise as the ultimate colonic delivery system (Basit, 2005).

Over the years, there are a number of patents applied to other colonic delivery system or related subjects. The following is a list briefly describing these various patents, which have been applied to colonic delivery or related matters in various aspects:
U.S. Pat. No. 4,627,851, issued to Wong et al in 1986, discloses a colonic-therapeutic delivery system that comprises three laminae: an inner semi-permeable lamina, a middle lamina containing a salt of a fatty acid, and an outer enteric lamina.
U.S. Pat. No. 4,910,021, issued to Davis et al in 1990, discloses a targeted enteral delivery system using an enteric coating that is consisted of aromatic carboxylic acids and their salts.
U.S. Pat. No. 5,171,580, issued to Iamartino et al in 1992, discloses an orally-pharmaceutical preparation with colon selective delivery with three protection layers at different solubility.
U.S. Pat. No. 5,346,703 and 6,346,272, issued to Viegas et al in 1994 and 2002 respectively, discloses a body cavity drug delivery with thermo-irreversible polyoxyalkylene and ionic polysaccharide gels in the presence of a counter-ion.
U.S. Pat. No. 5,415,864, issued to Kopecek et al in 1995, discloses a colonic-targeted oral drug-dosage forms based on crosslinked hydrogels containing azobonds and exhibiting pH-dependent swelling.
U.S. Pat No. 5,525,634 and U.S. Pat. No. 5,866,619, issued to Sintov et al in 1996 and 1999 respectively, discloses a colonic delivery system using a saccharide-containing polymer that is made of oligosaccharides and modified mucopolysaccharides.
U.S. Pat. No. 5,536,507, issued to Abramowitz et al in 1996, discloses a colonic drug delivery system using a three component formulations of which the first component is the drug, the second one is a delayed release coating, and the third one is an enteric coating.
U.S. Pat. No. 5,656,894, issued to Friend et al in 1997, disclosed a colonic delivery of drugs using hydrocolloid gum from higher plants and a pharmaceutically acceptable binder.
U.S. Pat. No. 5,686,105, issued to Kelm et al in 1997, discloses a pharmaceutical dosage form with two layers of enteric polymer coatings of which the inner layer and the outer layer are dissolved at different pH values.
U.S. Pat. No. 5,688,931, issued to Nogusa et al in 1997, discloses a drug carrier comprising a polysaccharide and a short peptide conjugates for high accumulation in a tumor.
U.S. Pat. No. 5,814,336, issued to Kelm et al in 1998, discloses a pharmaceutical dosage form for colonic delivery using an enteric polymer coating material that is dissolved in aqueous media at pH between about 5 to about 6.3.
U.S. Pat. No. 6,166,044, issued to Sandborn et al in 2000, discloses a method to treat inflammatory bowel disease by locally administering nicotine to the colon via rectal administration.
U.S. Pat. No. 6,228,396, issued to Watts in 2001, discloses a colonic drug delivery composition containing a starch capsule and a drug.
U.S. Pat. No. 6,413,494, issued to Lee et al in 2002, discloses a composition and pharmaceutical dosage form for colonic drug delivery using polysaccharides without a cross-linking agent.
U.S. Pat. No. 6,607,751, issued to Odidi et al in 2003, discloses a controlled delivery device for pharmaceutical agents incorporating xanthan gum
U.S. Pat. No. 7,001,888, issued to Tidmarsh et al in 2006, discloses methods and compositions for the treatment of cancer that take advantage of the increased uptake of glucose-anti-neoplastic agent conjugates in cancer cells.

However, none of the inventions and patents mentioned above, taken either singly or in any combination, is seen to describe the present invention as claimed. To the best of the inventors' knowledge, there are no existing patents ever issued which specifically disclose a method and system of using the galactose residue of polysaccharides from natural gums or selected plant materials chemically linked to a parent compound of anticancer drugs, steroids, antibiotics, and so on to synthesize a family of prodrugs with enhanced colonic delivery advantage like the current invention.

Accordingly, there is a need in the field to invent such a family of products. This invention therefore describes the method and process for preparing a novel family of polysaccharide prodrugs with enhanced colonic delivery advantage. This novel family of prodrugs is synthesized by chemically linking a parent compound with a specially selected polysaccharide with molecular weight of 10⁵-10⁷ Da containing galactose residues. Its distinctive characteristics are that it is a family of prodrugs synthesized by chemically linking polysaccharides with the parent compound through different bridge links for targeted colonic delivery; that the polysaccharides in the chemical compound contain galactose residues; and that these polysaccharides are prepared from natural gums or plant materials.

Due to these unique characteristics, the polysaccharide component of this family of novel prodrugs can protect the parent compound from absorption (or metabolism) in the upper gastrointestinal tract and deliver a high concentration of the bound compound to the colon area. Upon reaching the colon area, the active component of the parent drug will be released locally from the polysaccharide via enzymatic hydrolysis from the local bacterial flora, allowing it to act locally for colonic disease such as malignancy, inflammation or infection and/or taking advantage of the favorable microenvironment in the colon for steady and stable colonic drug absorption.

In the case of an oncology-prodrug, it can also take advantage of the specific galectin-3 binding property as galectin-3 is highly expressed in colorectal cancer cells (Schoeppner et al. 1995). Overall, this novel family of colon delivery target prodrugs can enhance the selectivity of the parent compound to reach the colon for either local action or enter the systemic circulation via the colonic uptake. Besides colorectal cancer, this may be desirable for various lower gastrointestinal diseases (Crohn's disease, ulcerative colitis, infectious gastrointestinal ailments) as well as for drugs that could benefit from a stable microenvironment for absorption.

In addition, many polysaccharides also have immunoregulation function along with some anti-tumor effect. This may be able to help enhance the efficacy of the parent compound. This invention therefore combines the medical design concepts of drug delivery, targeting, and synergism to achieve the goal of high efficacy and low toxicity.

As can be seen from the examples enclosed herein, this novel family of polysaccharide-based prodrugs possesses enhanced target specificity to colon cells. This unique property of the invention can lead to a higher efficacy locally or take advantage of the relative stable colonic microenvironment for steady systemic absorption, thus providing a preferential method to deliver the parent compound.

### SUMMARY OF THE INVENTION

This invention adopts the “Bacterial Triggering and Colon Targeting Drug Delivery System” theory as guidance utilizing prodrug technique to couple polysaccharides containing galactose with a parent compound through different bridge linkage to derive conjugates (Huang et al, 2002; Li et al, 2003). Because of this specific linkage, the family of prodrugs cannot be digested or hydrolyzed in the upper gastrointestinal tract, and therefore is delivered specifically to the colon. Once arriving at the colon, these prodrugs can be hydrolyzed by the bacterial enzyme in the lower intestinal tract to release the parent-compound-galactose, which can be used to achieve targeting action at the colonic cells.

Therefore, using large molecules containing galactose residues as the carrier of a parent compound will not only achieve locally release of drug in the colon, but also have the targeting effect specifically at the colonic cells, resulting in enhanced therapeutic effect of the parent compound. With increased selectivity, reduction in dosage is feasible and thus a potentially improved safety profile. This invention therefore combines the medical design concepts of drug delivery, targeting, and synergism to achieve the goal of high efficacy and low toxicity.

In addition, due to the lack of digestive enzymes in the colon, less intense motility, longer retention time, and higher responsiveness to enhancing agents, the colon represents a relatively more favorable microenvironment for drug absorption, compared to the stomach and small bowel, especially for poorly absorbed drugs. This more uniform environment will allow stable and homogeneous drug absorption. The current invention will allow the parent-compounds be delivered to the colon without degradation or metabolism in the upper GI tract to leverage on this advantage.

### BRIEF DESCRIPTION OF THE DRAWING

Other objects, features and advantages will be apparent from the following detailed descriptions of preferred embodiments taken in conjunction with the accompanying drawings in which:

FIG. 1. For illustration purposes only, an embodiment of the inventive drug delivery system wherein the galactose-containing polysaccharide is pectin, and Z refers to 5-FU. The symbols “**” and “*” indicate the position of β(1-4) glycosidic linkages, and n is from 1 to about 25,000.

### DETAILED DESCRIPTION OF THE INVENTION

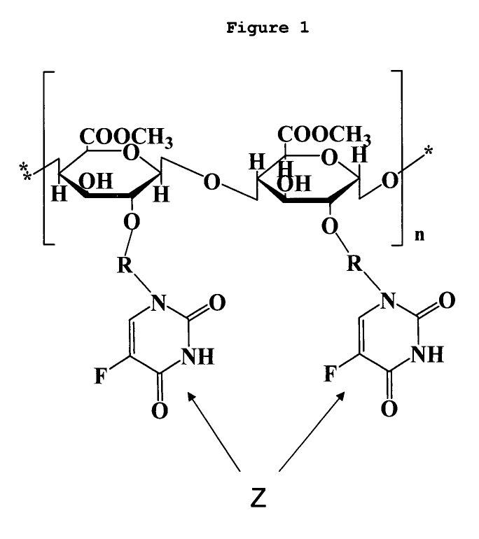

The purpose of this invention is to develop a method and process for preparing a family of prodrugs for enhanced colonic delivery and its application as indicated in FIG. 1 where R is the linker and Z is a parent compound.

Definitions

The following terms are used as defined herein. The use of these terms does not preclude the use of other terms not defined herein that are essentially synonymous with the defined terms.

The term prodrug refers to a compound whose efficacy is greatly enhanced after one or more conversion step(s) that occurs in vivo after administering the compound to a subject or patient.

The term galactose-containing polysaccharide refers to a polysaccharide having at least one galactose residue. A galactose-containing polysaccharide may be naturally occurring or may be prepared by modifying a different polysaccharide. Further, a galactose-containing polysaccharide may comprise unmodified galactose residues or modified galactose-derived residues.

The term galactose-containing fragment refers to a portion of the galactose-containing polysaccharide that may arise from being acted on by various enzymes. Enzymes that will generate galactose-containing fragments are largely expected in the colon. These enzymes are largely bacterial in nature.

The term therapeutic parent compound refers to a compound having therapeutic and/or diagnostic properties in a form prior to its linkage to a galactose-containing polysaccharide. This term is synonymous with parent compound and parent therapeutic compound.

The term derivatize or derivatizing refers to modifying a compound, e.g., galactose-containing polysaccharide or a therapeutic parent compound, by adding one or more reactive groups to the compound by reacting the compound with a functional group-adding reagent. As used herein, the term also refers to the attachment of cross-linkers to the compounds. The cross-linkers may be bifunctional, thus reacting with both compounds. A cross-linker possesses spacer arms that vary in size in different cross-linking compounds. This may be useful if one elects to have a known fixed distance between the galactose-containing polysaccharide and therapeutic parent compound.

The term linkage or linking bond, refers to the covalent bond connecting, or linking, the galactose-containing polysaccharide and the therapeutic parent compound. This bond may be formed by attaching one or more functional groups to either of, or both of, the therapeutic parent compound and galactose-containing polysaccharide. The galactose-containing polysaccharide and/or the therapeutic parent compound may be derivatized by the addition of various functional groups.

The term conjugate as used herein refers to the prodrug of the structural formula galactose-containing polysaccharide-R-Z.

The purpose of this invention is to develop a method and process for preparing a family of prodrugs for enhanced colonic delivery and its application as indicated in FIG. 1 where R is the linker and Z is a parent compound. First, the prodrug directs the parent compound to the colon, and then the parent-compound-galactose is bound to an appropriate protein on the surface of the colonic cells. This design can increase the parent compound selectivity, enhance its therapeutic effects, and reduce systemic toxicities, as well as with improved systemic absorption (if so desired).

The technical proposal of this invention is a novel family of prodrugs with enhanced colonic delivery advantage and its application with the characteristic of the chemical formula as indicated in FIG. 1. The structure shown is the prodrug for the colonic delivery resulted from the linkage of polysaccharides with a parent compound through various bridge linkages. Several unique features are:
The polysaccharides are the polysaccharides with galactose residues.
The polysaccharides with galactose include natural gums.
The natural gums are pectin, guar gum, and carob bean gum, and the vegetative polysaccharides include aloe polysaccharide, medlar polysaccharide, and rhubarb polysaccharide.
The compound resulted is Polysaccharide-R-Z, in which R is a linkage that is cleavable by bacterial enzymes present in the colon. The cleavable linkage can be replaced with any one of the following functional groups: R=—(CH₂)ₙ—, —CO—, —CO(CH₂)ₙ—, —CO(CH₂)ₙCO—, and n=1, 2, 3, 4; and Z is a parent compound, includes but not limited to, anticancer drugs, steroids, non-steroidal anti-inflammatory drugs (NSAIDs), antibiotics, antidiabetic drugs, etc.
The pectin, guar gum, and carob bean gum are hydrolyzed first with alkali (pH=9-10) then with acid (pH=3-5), and precipitated in alcohol and dialyzed to produce natural gums of target molecular weights of 10⁵-10⁷ Da, containing galactose for linkage with the parent compound.

The extraction method for the aloe polysaccharide, medlar polysaccharide, and rhubarb polysaccharide is as follows: First, pulverize the aloe (or medlar, rhubarb) plant materials and boiled with ethanol for three eight-hour-periods. The components dissolved in ethanol were extracted. The residue was boiled with water for another three eight-hour-periods in order to extract polysaccharides. All the water extractions were then collected. The polysaccharide-enriched fractions were obtained by precipitation with 5 volumes of ethanol for 3 times. After removing proteins, dialysis, separate and purify with gel filtration chromatography, polysaccharide components are obtained with molecular weights of 10⁵-10⁷ Da. During the extraction process, the following analytical instrumentation and techniques are implemented: a). High-performance liquid chromatography (HPLC) for purity analysis; b). Ultraviolet (UV) and infrared spectroscopic identification for qualitative examination; c). Measurement of sugar and glycuronic acid contents respectively by vitriol-phenol and vitriol-carbazole methods; and d). Measurement of monosaccharide compositions of the polysaccharides of different molecular weights and their component ratio with chromatographic techniques and gas chromatography.

The synthetic method for the prodrug is to bind the parent compound at the free hydroxyl group of the galactose residues contained in the polysaccharide via the formation of an ester or ether linkage through modifying the free hydroxyl group to a reactive carboxyl group (e.g. an acyl chloride). Then, the —NH₂ or similar functional groups (including —NHNH₂, —ONH₂, —NHC═(O)NHNH₂, —OH, —CO₂H, and —SH) of the parent compound reacts the modified carboxyl group of the galactose residues contained in the polysaccharides via the formation of an ester or ether linkage.

The following is but one illustrative embodiment of a method for linking the galactose-containing polysaccharide with a parent therapeutic compound, Z. A hydroxyl group in the 2 position of galactose is activated by chloroacetic acid. Then the activated carbonyl linker group reacts with the amine group of Z. This synthesis is exemplified below, using 5-FU as the embodiment of Z.

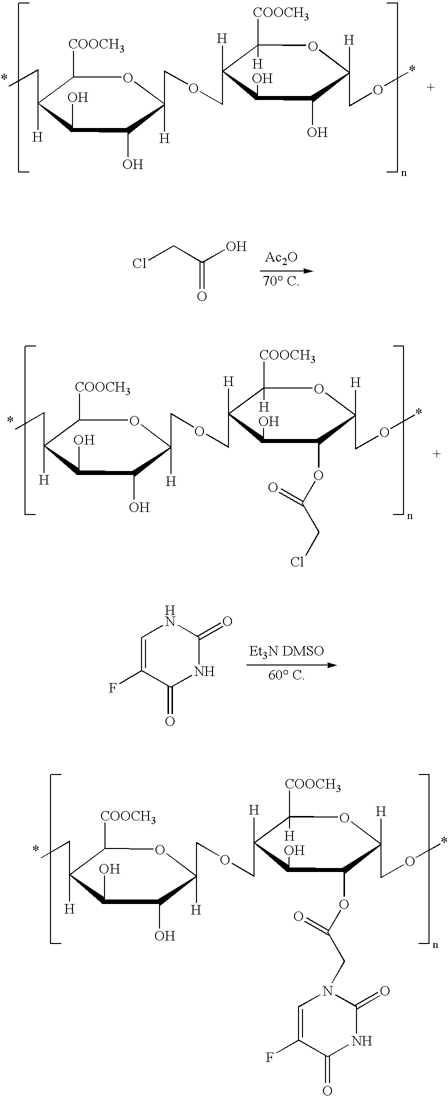

Alternatively, an amine group in an appropriate embodiment of Z reacts with chloroacetic acid to create a carboxylic acid linker group. Subsequent DCC (dicyclohexylcarbodiimide) coupling will link Z's newly added carboxylic acid linker group to the hydroxyl group in the 2 position of galactose in a galactose-containing polysaccharide. This method of linking is shown below.

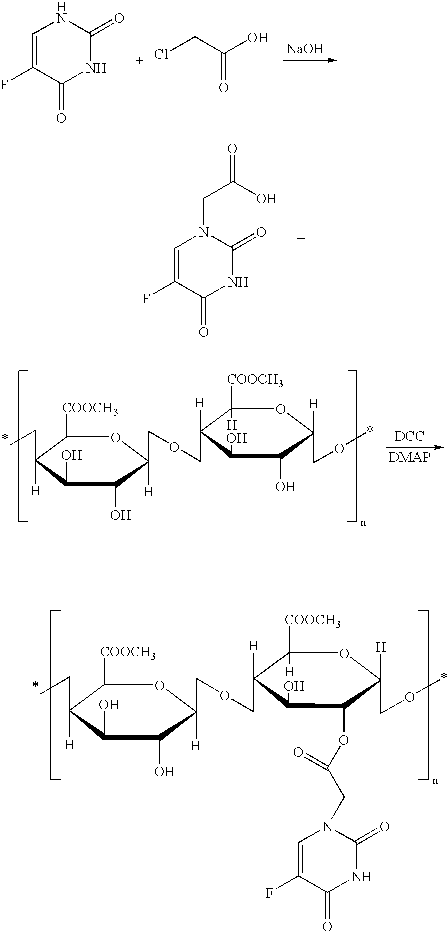

The formation of the ester linkage is made through acyl chloride method or N,N′-dicyclohexylcarbodiimide (DCC) method. The formation of the ester linkage is carried out through condensation. The formation of the acyl-amine linkage is derived from aminolysis of acyl chloride.

An embodiment of the drug delivery system involved in this invention is as follows:

Pectin: a polysaccharide composed of straight chains of galacturonic acid. The symbols “**” and “*” indicate the position of β(1-4) glycosidic linkages and n is from 1 to about 12,500.

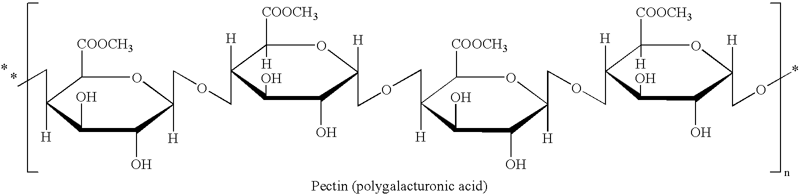

Guar Gum: a non-ionic polysaccharide mainly polymerized with galactose and mannose, belonging to natural galactomannan with mannose as its main chain and β(1-4) glycoside link as the linkage between D-mannopyranose units. Meanwhile, galactopyranose is connected to the mannose main chain through α(1-6) link. The molar ratio between mannose and galactose is 2:1.

Carob Bean Gum: a colorless and flavorless polysaccharide refined from plant endosperm, mainly containing mannose and galactose with an average molecular weight of 300 kDa.

It is currently known that the natural occurring gums containing galactose residues, such as pectin, guar gum, and carob bean gum have the functions of regulating the bacterial colonies in the intestinal tract as well cholesterol lowering. In addition, aloe polysaccharides, medlar polysaccharides, and rhubarb polysaccharides are rich in galactose with known immunoregulation functions, which have not, as of yet, been fully explored for pharmaceutical development.

The preparation methods of the prodrug involved in this invention and its characteristic of release in the colon will be described below. The new uses of the current colonic drug delivery and potential therapeutic use will also be discussed. However, this invention is not limited to the examples described below.

Preparation Method of the Novel Family of Prodrugs for the Targeted Delivery in the Colon

The natural gums containing galactose residues are hydrolyzed first with alkali (pH=9-10) then with acid (pH=3-5), and precipitated with alcohol and dialyzed to obtain natural gums of targeted molecular weights (10⁵-10⁷ Da) containing galactose residues.

The extraction method for galactose such as aloe polysaccharide, medlar polysaccharide, and rhubarb polysaccharides is to pulverize aloe, medlar, and rhubarb plant materials, and boiled with ethanol for three eight-hour-periods. The components dissolved in ethanol were extracted. The residue was boiled with water for another three eight-hour-periods in order to extract polysaccharides. All the water extractions were then collected. The polysaccharide-enriched fractions were obtained by precipitation with 5 volumes of ethanol for 3 times. After removing proteins, dialysis, separate and purify with gel filtration chromatography, polysaccharide components are obtained with molecular weights of 10⁵-10⁷ Da. During the extraction process, HPLC for purity analysis, UV and infrared spectroscopic identification for qualitative examination, measurement of sugar and glycuronic acid contents respectively by vitriol-phenol and vitriol-carbazole methods, and measurement of monosaccharide compositions of the polysaccharides of different molecular weights and their component ratio with chromatographic techniques and gas chromatography are performed.

Link the above-mentioned polysaccharides (including those prepared from natural gums) with the parent compound. The linkage method first changes the free hydroxyl group to reactive carboxyl group of the aforementioned polysaccharides and then connecting them with the parent compound under different conditions as per Implementation Example 1; the linkage method can also be modifying the parent compound first, and then connect it with the aforementioned polysaccharides under different conditions as per Implementation Examples 2 and 3.

This method includes connecting the parent compound with the hydroxyl group of polysaccharides through derivation to form an ester or ether linkage, or chemically linking polysaccharides with the —NH part of the parent compound to form an acyl-amine linkage through derivation. The formation of the ester linkage is carried out through acyl chloride method or N,N′-dicyclohexylcarbodiimide (DCC) method. The forming of the ester linkage is carried out through condensation, and the formation of the acyl-amine linkage is derived from aminolysis of acyl chloride.

### ILLUSTRATIVE EMBODIMENTS

In view of the foregoing disclosure several embodiments of the prodrug and its methods of preparation are disclosed. The following embodiments are presented for illustrative purposes only and are not meant to limit the scope of the claimed subject matter. Persons of ordinary skill in the art may be able to describe further embodiments based on the guidance set forth in the foregoing disclosure, the examples below and knowledge in the art.

A desirable embodiment is a prodrug that can deliver a therapeutic parent compound with target specificity toward the colon.

An additional embodiment is a prodrug capable of delivering a therapeutic parent compound to cells and/or tumors expressing the galactose binding lectin, galectin-3.

Embodiments directed to the methodologies for preparing and using the prodrug are also disclosed herein and are encompassed by invention.

An additional embodiment is encompassed and illustrated by the isolation of the appropriate galactose-containing polysaccharides and their linkage to a parent therapeutic compound.

Further desirable embodiments are encompassed and illustrated by the prodrug having a structural formula, polysaccharide-R-Z, wherein R is a covalent linkage and Z is a therapeutic parent compound.

Further desirable embodiments are encompassed and illustrated by the prodrug having a structural formula, polysaccharide-R-Z, wherein R is a covalent linkage and Z is an anti-cancer drug, a non-steroidal anti-inflammatory agent, a steroid, an antibiotic, an anti-diabetes agent, etc.

It is also desirable to provide embodiments of a prodrug for the targeted treatment of galectin-3 expressing cancers wherein the galactose-containing polysaccharide is prepared from natural gums or plant material. These embodiments of the prodrug may have a galactose-containing polysaccharide prepared from pectin, guar gum, and carob bean gum, and the plant materials aloe, medlar and rhubarb. However, virtually any plant material having galactose-containing polysaccharides would make a suitable starting material for isolating said galactose-containing polysaccharide.

Additional embodiments of the prodrug for targeting galectin-3 expressing cancers may have the parent compound 5-FU linked to a galactose-containing polysaccharide. The linkage, e.g., between 5-FU and a galactose-containing polysaccharide that is mediated by a bifunctional cross-linker having spacer-arms of varying lengths. Alternatively, linkages may be formed between a derivatized or underivatized galactose-containing polysaccharide and a derivatized or underivatized 5-FU. In other embodiments of the methods for preparing the prodrug the 5-FU may be derivatized. It is understood that derivatizing as referred to herein describes the addition of reactive groups to a galactose-containing polysaccharide or a 5-FU molecule without introducing the spacer arms that characterize commercially available cross-linkers.

Additional embodiments of the prodrug Polysaccharides-R-Z are encompassed and illustrated by a covalent linkage comprising any of the following functional groups: —(CH₂)ₙ—, —CO—, —CO(CH₂)ₙ—, —CO(CH₂)ₙCO—, and n=1, 2, 3, or 4.

An embodiment of the prodrug may e.g., result from forming a covalent linkage between the 5-FU and free hydroxyl groups of the galactose residues in the polysaccharides. This linkage may be achieved via the formation of ester or ether linkages through derivatization. An illustrative example would be to form the bond between the —NH of 5-FU at the free hydroxyl groups of the galactose residues in the polysaccharides via the formation of an acylamide linkage.

Embodiments of the methods for preparing the prodrug may use as starting material for galactose-containing polysaccharide isolation, pectin, guar gum, or carob bean gum. Either material is first hydrolyzed with alkali (pH=9-10) then with acid (pH=3-5), and followed by precipitation with alcohol and dialysis. These methods yield galactose-containing polysaccharides of molecular weights from approximately 10⁵ Da to approximately 10⁷ Da.

Additional embodiments of the methods for preparing the prodrug may comprise isolating the galactose-containing polysaccharides from aloe, medlar or rhubarb as follows: pulverizing the aloe/medlar/rhubarb plant material, and boiling with ethanol for three eight-hour-periods. The components dissolved in ethanol are extracted. The ethanol insoluble residue is boiled with water for another three eight-hour-periods in order to extract polysaccharides. All the water extractions are finally collected. The polysaccharide-enriched fractions are obtained by precipitation with 5 volumes of ethanol for 3 times. After removing proteins, dialysis, separate and purify with gel filtration chromatography, polysaccharide components are obtained with molecular weights of 10⁵ to 10⁷ Da. During the extraction process, high-performance liquid chromatography (HPLC) for purity analysis, ultraviolet (UV) and infrared spectroscopic identification for qualitative examination, measurement of sugar and glycuronic acid contents respectively by vitriol-phenol and vitriol-carbazole methods, and measurement of monosaccharide compositions of the polysaccharides of different (weight-average) molecular weights and their component ratio with chromatographic techniques and gas chromatography are performed.

Additional embodiments of the invention encompass linking a galactose-containing polysaccharide and 5-FU by the formation of the ester linkage made through acyl chloride method or N,N′-dicyclohexylcarbodiimide (DCC) method.

The embodiments of the prodrug illustrated above are effective for the treatment of galectin-3 expressing cancers, including but not limited to the following: breast, lung, prostate, bladder, thyroid, other head and neck, lymphoma, colon, pancreas and other gastrointestinal cancers.

The examples described below provide illustrative embodiments of methods of preparing the inventive prodrug. It should be readily appreciated that these examples taken together with knowledge in the art would allow persons in the art to practice related embodiments that are clearly encompassed by the subject matter disclosed and claimed herein.

### EXAMPLES

### Example 1

### Anti-Cancer Drugs

A prodrug with target specificity against colorectal cancer and its preparation methods. The major characteristic of this novel compound is that it is a prodrug synthesized by chemically linking a uniquely prepared polysaccharide with 5-fluorouracil (5-FU), irinotecan, capecitabine, and camptothecin through various bridge links. The R group in the following examples is galactose residues with linker groups.

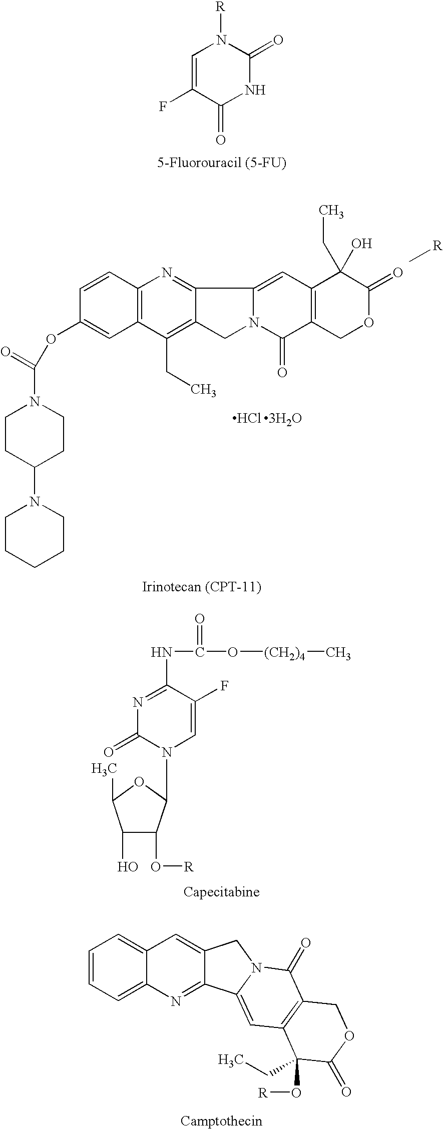

### Example 2

### Anti-Inflammatory Drugs—Steroids (S) and Non-Steroids (NS)

To treat inflammatory bowel disease, ulcerative colitis, and Crohn's disease, steroids, such as dexamethasone, hydrocortisone, prednisolone, and fluorocortisone, can be modified to become new prodrugs to achieve colonic specificity. Non-steroid anti-inflammatory drugs (NSAIDs), such as aspirin and ibuprofen. There are actually many NSAIDs (more than 10), aspirin and ibuprofen are included as illustrative examples only. The R group in the following examples is galactose residues with linker groups.

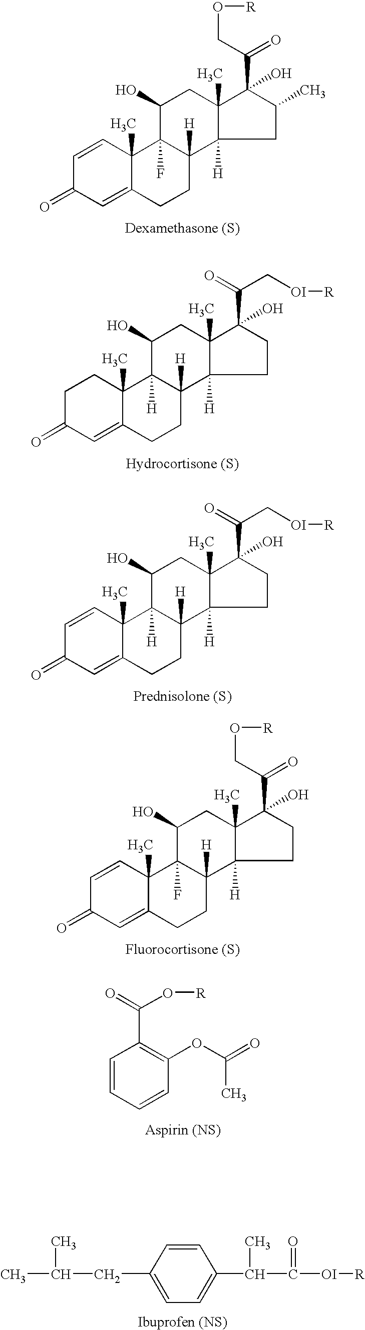

### Example 3

### Antibiotics

To achieve targeted colonic delivery and reduce side effects of antibiotics, drugs like penicillins, cephalosporins, quinolones, and metronidazole, can be linked to polysaccharides. The R group in the following examples is galactose residues with linker groups.

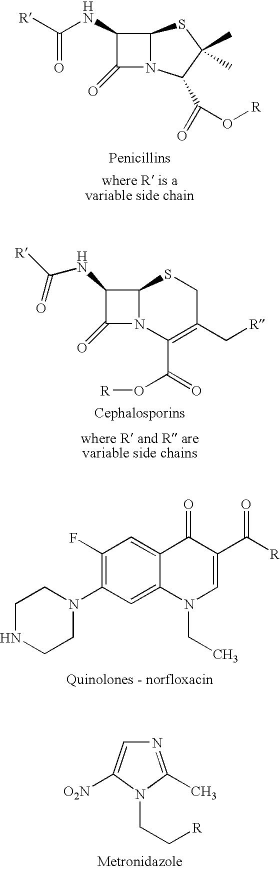

### Example 4

### Anti-Diabetic Drugs

To achieve colonic delivery and minimize side effects of other drugs that can benefit from colon delivery. One example is the anti-diabetic drugs in the class of biguanides, sulfonylurea, thiazolidinedione, etc can be linked to polysaccharides. The R group in the following examples is galactose residues with linker groups.

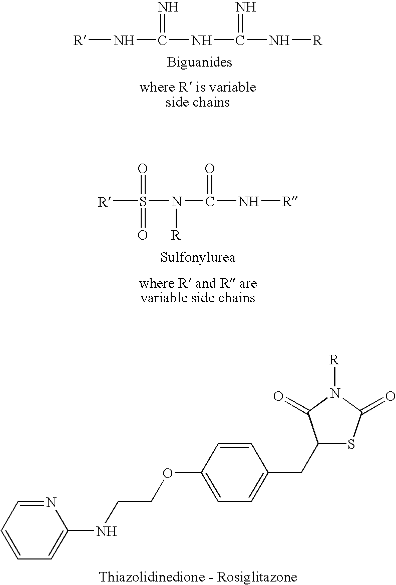

### Example 5

### Miscellaneous

To achieve systemic absorption via colon, any protein-based drug, vaccine or drug with poor upper GI absorption can be linked to polysaccharides. Vaccine prod-drugs via colonic delivery have the added advantage in that the colonic environment is rich in lymphoid tissues with abundant immune cells. Such approach can have the advantage of enhanced immune responses for the vaccine products.

This invention is not limited to the implementation examples as described in these specifications. The implementation examples are for illustration only. The actual pharmaceutical forms of this invention can be any suitable formulation in any vehicle to be used for patients.

Without intent to limit the scope of the invention, exemplary methods and their related results according to the embodiments of the present invention are given above. Note that titles or subtitles are used in the examples for convenience of a reader, which in no way should limit the scope of the invention. Moreover, certain theories are proposed and disclosed herein; however, in no way they, whether they are right or wrong, should limit the scope of the invention.

### REFERENCES

### U.S. Patent Documents

4,627,851 Dec. 9, 1986 Wong et al.
4,910,021 Mar. 20, 1990 Davis et al.
5,171,580 Dec. 15, 1992 Lamartine et al.
5,346,703 Sep. 13, 1994 Viegas et al.
5,415,864 May 16, 1995 Kopecek et al.
5,525,634 Jun. 11, 1996 Sintov et al.
5,536,507 Jul. 16, 1996 Abramowitz et al.
5,656,294 Aug. 12, 1997 Friend et al.
5,686,105 Nov. 11, 1997 Kelm et al.
5,688,931 Nov. 18, 1997 Nogusa et al.
5,814,336 Sep. 29, 1998 Kelm et al.
5,866,619 Feb. 2, 1999 Sintov et al.
6,166,044 Dec. 26, 2000 Sandborn et al.
6,228,396 May 8, 2001 Watts
6,346,272 Feb. 12, 2002 Viegas et al.
6,413,494 Jul. 2, 2002 Lee et al.
6,607,751 Aug. 19, 2003 Odidi et al.
7,001,888 Feb. 21, 2006 Tidmarsh et al.

### Foreign Patent Documents

WO 89/08119 September, 1989 WO.
WO 99/20316 April, 1999 WO.
WO 99/20316 April, 1999 WO.
WO 02/58741 August, 2002 WO.

### OTHER REFERENCES

1. Abu Shamat M. The role of gastrointestinal microflora in the metabolism of drugs. Int J Pharm 1993; 97:1-13.
2. Ashford M. Fell J T. Attwood D. et al. An in vivo investigation into the suitability of pH-dependent polymers for colonic targeting. Int J Pharm 1993; 95: 193-199.
3. Basit A W. Advances in Colonic Drug Delivery. Drugs. 65(14):1991-2007, 2005.
4. Cummings J H. Macfarlane G T. Drasar B S. The gut microflora and its significance. In: Whitehead R., editor. Gastrointestinal and oesophageal pathology. Edinburgh: Churchill Livingstone, 1989:201-219.
5. Davis S S. Hardy J G. Fara J W. Transit of pharmaceutical dosage forms through the small intestine. Gut 1986; 27:886-892.
6. Davis S S. Hardy J G. Stockwell A. et al. The effect of food on the gastrointestinal transit of pellets and an osmotic device (Osmet). Int J Pharm 1984;21-331-340.
7. Devereux J E. Newton J M. Short M B. The influence of density on the gastrointestinal transit of pellets. J Pharm Pharmacol 1990; 42:500-501.
8. Dew M J. Hughes P J. Lee M G. et al. An oral preparation to release drugs in the human colon. Br J Clin Pharmacol 1982; 14: 405-408.
9. Evans D F. Pye G. Bramley R. et al. Measurement of gastrointestinal pH profiles in normal ambulant human subjects. Gut 1988; 29: 1035-1041.
10. Finegold S M. Sutter V L. Mathisen G E. Normal indigenous intestinal flora. In: Hentges D J., editor. Human intestinal flora in health and disease. New York: Academic Press, 1983:3-13.
11. Gibson St. McFarlane C. Hay S. et al Significance of microflora in proteolysis in colon. Appl Environ Microbiol 1989; 55: 679-683.
12. Hu Z P. Mawatari S. Shibata N. et al. Application of a biomagnetic measurement system (BMS) to the evaluation of gastrointestinal transit of intestinal pressure-controlled colon delivery capsules (PCDCs) in human subjects. Pharm Res 2000; 7:160-167.
13. Hu Z P. Mawatari S. Shimokawa T. et al. Colon delivery efficiencies of intestinal pressure-controlled colon delivery capsules prepared by a coating machine in human subjects. J Pharm Pharmacol 2000; 52:1187-1193.
14. Huang J. Gao C S. Mei X G. New Type of Colon Targeting Drug Delivery System. Foreign Medical Sciences Section on Pharmacy. 29(5):306-308 & 320, 2002. (Publication in Chinese)
15. Ishibashi T. Pitcairn G R. Yoshino H. et al. Scintigraphic evaluation of a new capsule-type colon specific drug delivery system in healthy volunteers. J Pharm Sci 1998; 87:531-535.
16. Kaus L C. Fell J T. Sharma H. et al. On the intestinal transit of a single nondisintegrating object. Int J Pharm 1984; 14:143-148.
17. Khosla R. Davis S S. Gastric emptying and small and large bowel transit of nondisintegrating tablets in fasted subjects. Int J Pharm 1989; 52: 1-10.
18. Li G F. Chen J H. Liu L J. Guo D. Yan Y. Xu Z Y. Chen Z L. Absorption and Distribution of 5-Aminosalicylic Acid from its Chitosan Capsule Degraded by Colon Bacteria-Released Enzymes in Rats. Journal of First Military Medical School. 23(5):431-434, 2003. (Publication in Chinese)
19. Luo Y. Zhuo R X. Fan C L. Studies on the Synthesis and Antitumor Activity of Biodegradable Polyphosphamides. Chemical Journal of Chinese Universities. 15(5):767-770, (Publication in Chinese)
20. Nugent S G. Kumar D. Rampton D S. Et al. Intestinal luminal pH in inflammatory bowel disease: possible determinants and implications for therapy with aminosalicylates and other drugs. Gut 2001; 48:571-577.
21. Ohya Y. Inosaka K. Ouchi T. Synthesis and antitumor activity of 6-O-carboxymethyl chitin fixing 5-fluorouracils through pentamethylene, monomethylene spacer groups via amide, ester bonds. Chemical & Pharmaceutical Bulletin. 40(2):559-61, 1992.
22. Ohya Y. Takei T. Kobayashi H. Ouchi T. Release behavior of 5-fluorouracil from chitosan-gel microspheres immobilizing 5-fluorouracil derivative coated with polysaccharides and their cell specific recognition. Journal of Microencapsulation. 10(1):1-9, 1993.
23. Ouchi T. Hagihara Y. Takahashi K. Takano Y. Igarashi I. Synthesis and antitumor activity of poly(ethylene glycol)s linked to 5-fluorouracil via a urethane or urea bond. Drug Design & Discovery. 9(1):93-105, 1992.
24. Schoeppner H L. Raz A. Ho S B. Bresalier R S. Expression of an endogenous galactose-binding lectin correlates with neoplastic progression in the colon. Cancer. 75(12):2818-26, 1995.
25. Tuleu C. Basit A W. Waddington W A et al. Colonic delivery of 4-aminosalicylic acid using amylose-ethylcellulose coated hydroxypropyl methylcellulose capsules. Aliment Pharmacol Ther 2002; 167: 1771-1779.
26. Van den Mooter G. Stas G. Damian F. et al. Stability of UC-781, in intestinal mucosal homogenates of the rat, rabbit, and pig. Pharm Res 1998; 15;11:1799-1802.
27. Wilding I R. The enterion capsule: a novel technology for understanding the biopharmaceutical complexity of new molecular entities (NMEs). Drug Deliv Tech 2001; 1(1): 8-11.
28. Youan B B C. Chronopharmaceutics: gimmick or clinically relevant approach to drug delivery. J Control Release 2004; 98:337-353.
29. Zhu K J. Zhang J X. Wang C. Yasuda H. Ichimaru A. Yamamoto K. Preparation and in vitro release behaviour of 5-fluorouracil-loaded microspheres based on poly (L-lactide) and its carbonate copolymers. Journal of Microencapsulation. 20(6):731-43, 2003.

What is claimed is:

1. A prodrug comprising,
a) a galactose-containing polysaccharide;
b) a therapeutic parent compound effective for treating colorectal cancer, inflammation, infection, or which can benefit from a colonic delivery system, and
c) a covalent bond connecting a) to b).

2. The prodrug of claim 1 wherein the galactose-containing polysaccharide comprises more than one galactose residue.

3. The prodrug of claim 1 wherein the galactose-containing polysaccharide has a molecular weight of about 10⁵ Da to about 10⁷ Da.

4. The prodrug of claim 1 wherein the parent compound comprises an oxygen, nitrogen or sulfur atom available for linkage to the galactose-containing polysaccharide.

5. A prodrug having the structural formula
polysaccharide-R-Z, wherein
Z comprises a therapeutic parent compound and R comprises a covalent bond linking Z to the polysaccharide, and wherein the polysaccharide is a galactose-containing polysaccharide.

6. The prodrug of claim 5, wherein R comprises an ester, an ether, an amide, an amine, a hydroxylamine, a thioether or thioester.

7. The prodrug of claim 5, wherein R comprises an ester, ether, an amide, an acyl amine or an amine.

8. The prodrug of claim 1, wherein the galactose-containing polysaccharide occurs naturally.

9. The prodrug of claim 1, wherein the galactose-containing polysaccharide and the therapeutic parent compound are linked by a covalent bond comprising a linkage selected from the group consisting of —(CH₂)ₙ—, —CO—, —CO(CH₂)ₙ—, and —CO(CH₂)ₙ—CO— and wherein n is from 1 to 4.

10. The prodrug of claim 1 having the structure shown in FIG. 1.

11. The prodrug of claim 1 or 5, wherein the galactose-containing polysaccharide, or a galactose-containing fragment thereof, is capable of binding to galectin-3.

12. The prodrug of claim 11 comprising at least one galactose-containing fragment to which the therapeutic parental compound is covalently linked.

13. The prodrug of claim 11, wherein the at least one galactose-containing fragment results from the action of bacterial enzymes that degrade the galactose-containing polysaccharide.

14. The prodrug of claim 13, wherein the galactose-containing fragment further comprises the parental therapeutic compound.

15. The prodrug of claim 13, wherein the bacterial enzymes that produce the galactose-containing fragment are in the colon.

16. The prodrug of claim 5 wherein Z is 5-fluorouracil (5-FU), irinotecan, capecitabine, or camptothecin.

17. The prodrug of claim 5 wherein Z is dexamethasone, hydrocortisone, prednisolone, or fluorocortisone.

18. The prodrug of claim 5 wherein Z is aspirin or ibuprofen.

19. The prodrug of claim 5 wherein Z is sulfonylurea, thiazolidinedione or a biguanide.

20. The prodrug of claim 5 wherein Z is a penicillin, a cephalosporin, a quinolone or metronidazole.

21. A method for preparing a prodrug having the structural formula
polysaccharide-R-Z, wherein
Z comprises a parent compound and R comprises a covalent bond connecting Z to the polysaccharide, and wherein the polysaccharide is a galactose-containing polysaccharide, the method comprising the steps of:
a) hydrolyzing pectin, guar gum and carob bean gum in alkali at a pH from about 9 to about 10;
b) hydrolyzing the product of step a) in acid at a pH from about 3 to about 5;
c) purifying the galactose-containing polysaccharide and
d) reacting the galactose-containing polysaccharide with a parent therapeutic compound Z, thereby forming covalent bond R comprising either an ester, an ether, an amide, an amine, an acyl amine a hydroxylamine, a thioester, or a thioether.

22. A method for preparing a prodrug having the structural formula
polysaccharide-R-Z, comprising the steps of,
a) pulverizing either aloe, medlar, or rhubarb and treating the pulverized material with ethanol to obtain a soluble phase and an insoluble residue;
b) extracting the insoluble residue in boiling water to obtain polysaccharides,
c) purifying the polysaccharide, and
d) reacting the polysaccharide with a therapeutic parent compound Z to form covalent bond R comprising either an ester, an ether, an amide, an amine, an acyl amine, a hydroxylamine, a thioester, or a thioether.

23. The method of claim 21 or 22 further comprising derivatizing the polysaccharide by addition of a functional group from the group consisting of ester, an ether, an amide, an amine, a hydroxylamine, a thioether or thioester.

24. The method of claim 21 or 22 wherein the added functional group forms a covalent bond with the parent compound, and the covalent bond comprises a linkage selected from the group consisting of —(CH₂)ₙ—, —CO—, —CO(CH₂)ₙ—, and —CO(CH₂)ₙ—CO—, wherein n is from 1 to 4.

25. The method of claim 21 or 22 wherein Z is 5-fluorouracil (5-FU), irinotecan, capecitabine, or camptothecin.

26. The method of claim 21 or 22 wherein Z is dexamethasone, hydrocortisone, prednisolone, and fluorocortisone.

27. The method of claim 21 or 22 wherein Z is aspirin or ibuprofen.

28. The method of claim 21 or 22 wherein Z is sulfonylurea, thiazolidinedione or a biguanide.

29. The method of claim 21 or 22 wherein Z is a penicillin, a cephalosporin, a quinolone or metronidazole.

30. A pharmaceutical composition comprising an effective amount of a prodrug having the structural formula polysaccharide-R-Z, comprising
a) a naturally occurring galactose-containing polysaccharide;
b) Z comprises a therapeutic parent compound and
c) R comprises a covalent bond connecting Z to the polysaccharide;
and a pharmaceutically suitable carrier, filler or adjuvant.

31. A pharmaceutical composition comprising an effective amount of a prodrug having the structural formula
polysaccharide-R-Z, comprising
a) a naturally occurring galactose-containing polysaccharide;
b) Z comprises 5-fluorouracil (5-FU), irinotecan, capecitabine, or camptothecin,
and
c) R comprises a —(CH₂)ₙ—, —CO—, —CO(CH₂)ₙ—, and —CO(CH₂)ₙ—CO—, wherein n is from 1 to 4,
and a pharmaceutically suitable carrier, filler or adjuvant.

32. The pharmaceutical composition of claim 30 wherein Z is 5-FU and R comprises a —(CH₂)ₙ—, —CO—, —CO(CH₂)ₙ—, and —CO(CH₂)ₙ—CO— wherein n is from 1 to 4.

33. The pharmaceutical composition of claim 31 wherein Z is 5-FU and R comprises a —CO(CH₂)ₙ—CO—, and wherein n is from 1 to 4.

34. The pharmaceutical composition of claim 31 wherein Z is 5-FU and R comprises a —CO(CH₂)ₙ—, and wherein n is from 1 to 4.

35. A colorectal-targeted prodrug comprising,
a) a galactose-containing polysaccharide;
b) a therapeutic parent compound that distributes locally and/or systemically upon release from the prodrug,
and
c) a covalent bond connecting a) to b).

36. A colorectal-targeted prodrug comprising,
a) a galactose-containing polysaccharide;
b) a therapeutic parent compound that distributes locally and/or systemically upon release from the prodrug in the colon,
and
c) a covalent bond connecting a) to b).

37. A prodrug comprising,
a) a galactose-containing polysaccharide;
b) a therapeutic parent compound selected from the group consisting of anti-cancer compounds, corticosteroids, non-steroidal anti-inflammatory compounds, and antibiotics; and
c) a covalent bond connecting a) to b),
wherein the therapeutic compound is released from the prodrug in the colorectal region of the gastrointestinal tract and is distributed either systemically and/or locally.
